(19) United States
(12) Patent Application Publication
(10) Pub. No.: US 2023/0240608 A1
(43) Pub. Date: Aug. 3, 2023

(54) IN-EAR ELECTRODES FOR AR/VR APPLICATIONS AND DEVICES

(71) Applicant: Meta Platforms Technologies, LLC, Menlo Park, CA (US)

(72) Inventors: Morteza Khaleghimeybodi, Bothell, WA (US); Barry David Silverstein, Kirkland, WA (US); Nils Thomas Fritiof Lunner, Redmond, WA (US); John Rumsfeld, San Francisco, CA (US); Andrew John Ouderkirk, Kirkland, WA (US); Joshua Weiner, Great Neck, NY (US)

(21) Appl. No.: 18/069,002

(22) Filed: Dec. 20, 2022

Related U.S. Application Data
(60) Provisional application No. 63/305,932, filed on Feb. 2, 2022.
(60) Provisional application No. 63/356,851, filed on Jun. 29, 2022.
(60) Provisional application No. 63/356,860, filed on Jun. 29, 2022.
(60) Provisional application No. 63/356,864, filed on Jun. 29, 2022.
(60) Provisional application No. 63/356,872, filed on Jun. 29, 2022.
(60) Provisional application No. 63/356,877, filed on Jun. 29, 2022.
(60) Provisional application No. 63/356,883, filed on Jun. 29, 2022.

(51) Int. Cl. A61B 5/00; A61B 5/349

(52) U.S. Cl. CPC A61B 5/6817; A61B 5/721; A61B 5/349; A61B 2562/0204

(57) ABSTRACT

A device for performing electrical measurements for in-ear monitoring is provided. The device includes an in-ear fixture configured to fit in an ear canal of a user, a first electrode mounted on the in-ear fixture and configured to receive an electronic signal from the skin, and an internal microphone to receive an acoustic signal, propagating through the ear of the user. The device also includes an external microphone coupled to receive an external acoustic signal, propagating through an environment, and a processor that is coupled to an augmented reality headset, the processor identifies a cardiovascular condition, or a neurologic condition of the user based on at least one of the electronic signals, the internal acoustic signal, and the external acoustic signal. A memory storing instructions which, when executed by a processor cause a method of use of the above device. The memory, the processor and the method are also provided.

### CROSS-REFERENCE TO RELATED APPLICATIONS

The present disclosure is related and claims priority under 35 U.S.C. § 119(e) to U.S. Prov. Appln. No. 63/305,932, entitled IN-EAR BIO-SENSING FOR AR/VR APPLICATIONS AND DEVICES, filed on Feb. 2, 2022, to U.S. Prov. Appln. No. 63/356,851, entitled IN-EAR ELECTRODES FOR AR/VR APPLICATIONS AND DEVICES, to U.S. Prov. Appln. No. 63/356,860, entitled IN-EAR OPTICAL SENSORS FOR AR/VR APPLICATIONS AND DEVICES, to U.S. Prov. Appln. No. 63/356,864, entitled IN-EAR MOTION SENSORS FOR AR/VR APPLICATIONS AND DEVICES, to U.S. Prov. Appln. No. 63/356,872, entitled IN-EAR TEMPERATURE SENSORS FOR AR/VR APPLICATIONS AND DEVICES, to U.S. Prov. Appln. No. 63/356,877, entitled IN-EAR MICROPHONES FOR AR/VR APPLICATIONS AND DEVICES, to U.S. Prov. Appln. No. 63/356,883, entitled IN-EAR SENSORS AND METHODS OF USE THEREOF FOR AR/VR APPLICATIONS AND DEVICES, all filed on Jun. 29, 2022, to Morteza KHALEGHIMEYBODI, et al., the contents of which applications are hereby incorporated by reference in their entirety, for all purposes.

### BACKGROUND

### Field

The present disclosure is related to in-ear electrodes for use in virtual reality and augmented reality environments and devices. More specifically, the present disclosure is related to electrodes configured to receive electrical signals inside the ear for health monitoring with in-ear devices for immersive reality applications.

### Related Art

Current in-ear devices (e.g., hearing aids, hearables, headphones, earbuds, and the like) for mobile and immersive applications are typically bulky and uncomfortable for the user. Adding health sensing capabilities to in-ear devices is hindered by the small form factors desirable in such devices and the complex data processing and analysis involved.

### SUMMARY

In a first embodiment, a device includes an in-ear fixture configured to fit in an ear canal of a user, a first electrode mounted on the in-ear fixture and configured to receive a first electronic signal from a skin in the ear canal of the user, an internal microphone coupled to receive an internal acoustic signal, propagating through the ear canal of the user, an external microphone coupled to receive an external acoustic signal, propagating through an environment of the user, and a processor that is coupled to an augmented reality headset, the processor configured to identify a cardiovascular condition, or a neurologic condition of the user based on at least one of the first electronic signal, the internal acoustic signal, and the external acoustic signal.

In a second embodiment, a computer-implemented method includes receiving, from a first electrode, a first electronic signal from a skin in a first ear canal of a user of an in-ear device, forming a waveform with the first electronic signal, and identifying one of a heart activity or a brain activity of the user based on the first electronic signal.

In a third embodiment, a non-transitory, computer-readable medium stores instructions which, when executed by a processor cause a computer to perform a method. The method includes receiving, from a first electrode, a first electronic signal from a skin in a first ear canal of a user of an in-ear device, forming a waveform with the first electronic signal, and identifying one of a heart activity or a brain activity of the user based on the first electronic signal.

In yet other embodiments, a system includes a first means to store instructions and a second means to execute the instructions to perform a method. The method includes receiving, from a first electrode, a first electronic signal from a skin in a first ear canal of a user of an in-ear device, forming a waveform with the first electronic signal, and identifying one of a heart activity or a brain activity of the user based on the first electronic signal.

These and other embodiments will become clear for one of ordinary skill in light of the following.

### BRIEF DESCRIPTION OF THE FIGURES

FIG. 1 illustrates an AR headset and an in-ear monitor (IEM) in an architecture configured to assess a user's health, according to some embodiments.

FIG. 2 illustrates an augmented reality ecosystem including wearable devices in the ear and wrist to assess a user's health, according to some embodiments.

FIGS. 3A-3D illustrate different embodiments of an in-ear monitor (IEM), according to some embodiments.

FIGS. 4A-4H illustrate multiple electrodes disposed across an in-ear device for electroencephalogram (EEG), electrocardiogram (ECG), electrodermal activity (EDA), and electrooculogram (EOG) sensing, according to some embodiments.

FIGS. 5A-5B illustrate EOG waveforms for gaze estimation with an in-ear device or IEM, according to some embodiments.

FIG. 6 illustrates an ECG waveform measured with an IEM, according to some embodiments.

FIG. 7 illustrates a gesture for an on-demand ECG capture system from an IEM device, according to some embodiments.

FIG. 8 illustrates an in-ear ECG waveform and its spectral decomposition, according to some embodiments.

FIG. 9 illustrates a hearing threshold estimation using an IEM, according to some embodiments.

FIG. 10 illustrates a block diagram of a conventional EEG readout with an instrumental amplifier (IA) and an active electrode (AE), according to some embodiments.

FIG. 11 is a flow chart illustrating steps in a method 1100 for using electrodes in an in-ear monitor for assessing the health of a user of a headset or smart glasses, according to some embodiments.

FIG. 12 is a block diagram illustrating an exemplary computer system with which headsets and other client devices, and the method in FIG. 11 be implemented, according to some embodiments.

In the figures, elements having the same or similar reference numeral have the same or similar features and attributes, unless explicitly stated otherwise.

### DETAILED DESCRIPTION

In the following detailed description, numerous specific details are set forth to provide a full understanding of the present disclosure. It will be apparent, however, to one ordinarily skilled in the art, that the embodiments of the present disclosure may be practiced without some of these specific details. In other instances, well-known structures and techniques have not been shown in detail so as not to obscure the disclosure.

### General Overview

Head-worn devices (i.e., devices worn on head including but not limited to hearables, smart glasses, AR/VR headsets and smart glasses, etc.) offer opportunities to access valuable health information.

The ear (e.g., the ear canal and ear concha and pinna) has close proximity to the brain, to body chemistry, and blood vessels indicative of brain activity and cardio-respiratory activity, and inner body temperature. More specifically, sensors can be placed inside the ear canal or around the ear (in the case of AR/VR headsets or smart glasses) to sense the brain's, heart's, and ocular electrophysiological activities (e.g., electro-encephalography EEG, electro-cardiography ECG, electro-oculography EOG, electrodermal activity EDA, and the like); or to sense vital signs (heart rate, breathing rates, blood pressure, body temperature, and the like); or to sense the body chemistry (e.g., blood alcohol level, blood glucose estimation, and the like).

Electrodes in embodiments as disclosed herein may be used in EOG, ECG, or EEG measurements, e.g., for determining auditory attention, heart rate estimation, breathing rate, and the like, Auditory Steady State Response —ASSR—, or auditory brainstem response —ABR—. In some embodiments, in-ear electrodes as disclosed herein may be useful to measure resting state electric oscillations (alpha waves in an EEG) that can track relaxation/activity. With the combination of other measurements (e.g., photoplethysmography, PPG), a new branch of diagnostic possibilities is open. In-ear EEG measurements can be applied to track user attention (e.g., distinguishing between attention focus from eye gaze direction).

Methods and devices disclosed herein include optical, acoustical, motion sensors, chemical sensors, and temperature sensors, in and around the ears of AR/VR headset users, in combination with software correlation of the signals provided by the above sensors to generate comprehensive diagnostics and health evaluation of the user.

Some of the features disclosed herein include in-ear or head-worn body temperature sensing using infrared sensing and spectroscopy techniques. In some embodiments, the contact area for sensors as disclosed herein include the in-ear canal (like an in-ear earbud) and within the conchal bowl (in human pinna), areas on top of the human ear (where the glasses sit), and areas in the nose-pad of a headset or smart glasses (where glasses sit on the nose). Some measurements may include in-ear or around the ear sensing of glucose level, alcohol sensing, body temperature, blood pressure, and the like. Some embodiments include opto-electrical-based pulse transit time (PTT) methodology to estimate blood pressure for a glasses/headset device using a combination of optical and electrical signals (e.g., PPG+ECG sensors respectively). Some embodiments include optical-based pulse transit time (PTT) methodology to estimate blood pressure for a glasses/headset device using a combination of optical signals collected from multiple different wavelength (e.g., using a PPG sensor with more than one distinct wavelength). Some embodiments obtain user's blood pressure using an optical sensing technique (PPG) in combination with a deep neural network to train a network based using both PPG information and a corresponding ground-truth blood pressure information. Some embodiments include motion-based pulse transit time (PTT) methodology to estimate blood pressure for a glass/headset device using a combination of motion sensor and electrical signals (e.g., IMU+ECG sensors respectively). Once fully trained, the neural network can then quantify and predict the user's blood pressure using just the PPG information and leveraging this pre-trained network. To further improve the accuracy, some subjective calibrations may be desirable. In some embodiments, PPG signals collected in IEM devices as disclosed herein may be able to estimate the cognitive load on the user with analysis of oxygenated and deoxygenated blood flow (oxy- and deoxy-hemoglobin) to the brain. Some embodiments include sensing alcohol levels through emissions around the ear. Some embodiments incorporate chemical sensing intake around the contact points of the ear. In some embodiments, IEM devices may perform alcohol monitoring and fat burning during user exercise.

### Example System Architecture

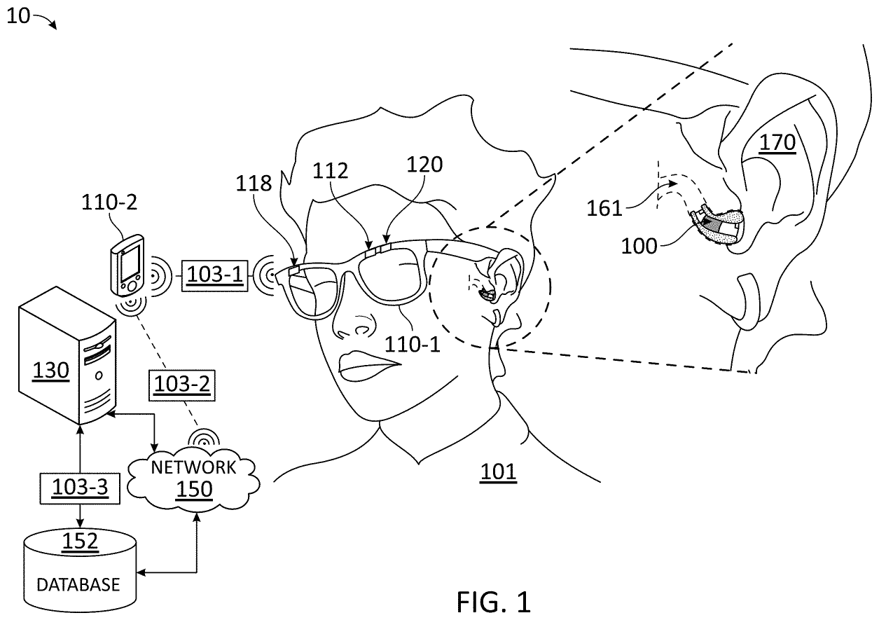

FIG. 1 illustrates an AR headset 110-1 and an in-ear monitor (IEM) 100 in an architecture 10 configured to assess the health of a user 101, according to some embodiments. IEM 100 is inserted in the ear 170 of user 101, reaching the ear canal 161. AR headset 110-1 may include smart glasses having a memory circuit 120 storing instructions and a processor circuit 112 configured to execute the instructions to perform steps as in methods disclosed herein. AR headset 110-1 (or smart glasses) may also include a communications module 118 configured to wirelessly transmit information (e.g., Dataset 103-1) between AR headset 110-1 (and/or in-ear device 100, and/or a smart watch, or combination of the above) and a mobile device 110-2 with the user (AR headset 110-1 and mobile device 110-2 will be collectively referred to, hereinafter, as “client devices 110”). Communications module 118 may be configured to interface with a network 150 to send and receive information, such as dataset 103-1, dataset 103-2, and dataset 103-3, requests, responses, and commands to other devices on network 150. In some embodiments, communications module 118 can include, for example, modems or Ethernet cards. Client devices 110 may in turn be communicatively coupled with a remote server 130 and a database 152, through network 150, and transmit/share information, files, and the like with one another (e.g., dataset 103-2 and dataset 103-3). Datasets 103-1, 103-2, and 103-3 will be collectively referred to, hereinafter, as “datasets 103.” Network 150 may include, for example, any one or more of a local area network (LAN), a wide area network (WAN), the Internet, and the like. Further, the network can include, but is not limited to, any one or more of the following network topologies, including a bus network, a star network, a ring network, a mesh network, a star-bus network, tree or hierarchical network, and the like.

In some embodiments, at least one of the steps in methods as disclosed herein are performed by processor 112, providing dataset 103-1 to mobile device 110-2. Mobile device 110-2 may further process the signals and provide dataset 103-2 to database 152 via network 150. Remote server 130 may collect dataset 103-2 from multiple AR headsets 110-1 and mobile devices 110-2 in the form and perform further calculations. In addition, having aggregated data from a population of individuals, the remote server may perform meaningful statistics. This data cycle may be established provided each of the users involved have consented for the use of de-personalized, or anonymized data. In some embodiments, remote server 130 and database 152 may be hosted by a healthcare network, or a healthcare facility or institution (e.g., hospital, university, government institution, clinic, health insurance network, and the like). Mobile device 110-2, AR headset 110-1, in-ear device 100, and applications therein may be hosted by a different service provider (e.g., a network carrier, an application developer, and the like). Moreover, AR headset 110-1 and mobile devices 110-2 may proceed from different manufacturers. User 101 is ultimately the sole owner of dataset 103-1 and all data derived therefrom (e.g., datasets 103), and so all the data flows (e.g., datasets 103), while provided, handled, or regulated by different entities, are authorized by user 101, and protected by network 150, server 130, database 152, and mobile device 110-2 for privacy and security.

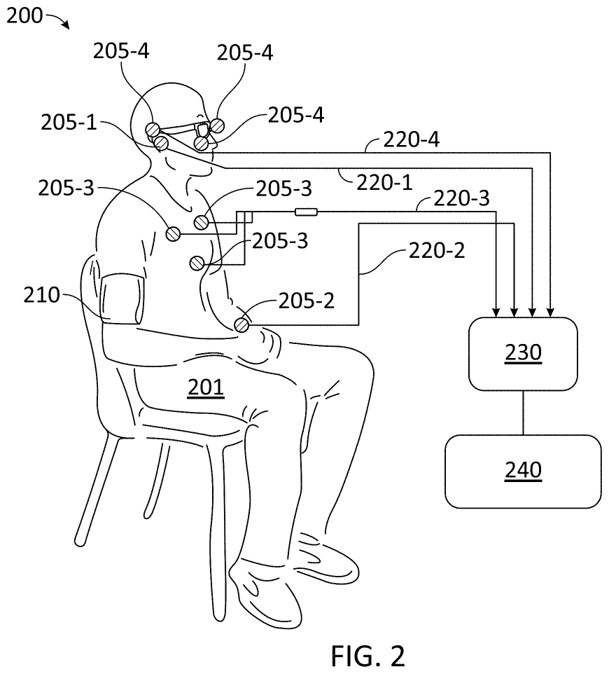

FIG. 2 illustrates an augmented reality ecosystem 200 including wearable devices in the ear 205-1 (e.g., an IEM), wrist 205-2, chest 205-3, and smart glass sensors 205-4 to assess the health of user 201, according to some embodiments. In some embodiments, IEM 205-1 further includes an optical sensor configured to provide an optical signal 220-1 to a processor in a computer 240 via a data acquisition module (DAQ) 230. IEM 205-1 may further include one or more contact electrodes configured to provide an electrical signal to a processor in a computer 240 via a data acquisition module (DAQ) 230. Computer 240 is configured to identify a cardiovascular condition of user 201 based on a first electronic signal from IEM 205-1 and optical signal 220-1. In some embodiments, IEM 205-1 further includes a motion sensor (e.g., an accelerometer, a contact microphone, or an IMU) configured to provide a motion-based signal to computer 240 via DAQ 230. In some embodiments, a pair of IEMs 205 will be placed in both ears and different optical, electrical (electrode), acoustic (microphone), or motion sensors (accelerometer, IMU, contact microphone, etc.) may be placed in either both sides; or in some cases, some sensors may be placed on one side (e.g., the Right side) and some other sensors may be placed on the other side (e.g., Left side). Computer 240 is configured to identify a cardiovascular condition of the user based on a first electronic signal from IEM 205-1 and the motion signal. The optical sensor may be a photo-plethysmography (PPG) sensor and optical signal 220-1 may include a digital or analog signal indicative of a vascular activity inside the ear of user 201. Chest sensors 205-3 and smart glass sensors 205-4 may include ECG sensors to provide a distributed signals 220-3 and 220-4 from one or more areas around the chest and face (e.g., the outside of the ear, the chin, and the nose) of user 201, respectively (or alternatively an ECG can be collected from some electrodes placed on areas on the head or from electrodes placed in IEM 205-1, or electrodes placed on the wrist device 205-2), and a wrist PPG sensor in device 205-2 may provide a separate signal 220-2 for vascular activity around the wrist of user 201. IEM 205-1, wrist sensor 205-2, chest sensors 205-3, and smart glass sensors 205-4 will be collectively referred to, hereinafter, as “wearable devices (and sensors) 205.” Blood pressure (BP) measurements may be obtained with a cuff or cuff-less BP monitor 210 and may also be determined by comparing PPG signals 220-1 and 220-2. Signals 220-1, 220-2, 220-3 and 220-4 (hereinafter, collectively referred to, hereinafter, as “signals 220”) may be collected and digitized by DAQ 230 in computer 240, for processing. In some embodiments, signals 220 and others may be wired, or wireless. In some embodiments, it may be preferable to have wireless signal communication between the different wearable devices 205 with user 201. In some embodiments, wearable devices and sensors 205 may include one or more motion sensors, and the motion-based information collected from the smart glass, the IEM, chest or wrist can be combined to create a more meaningful information.

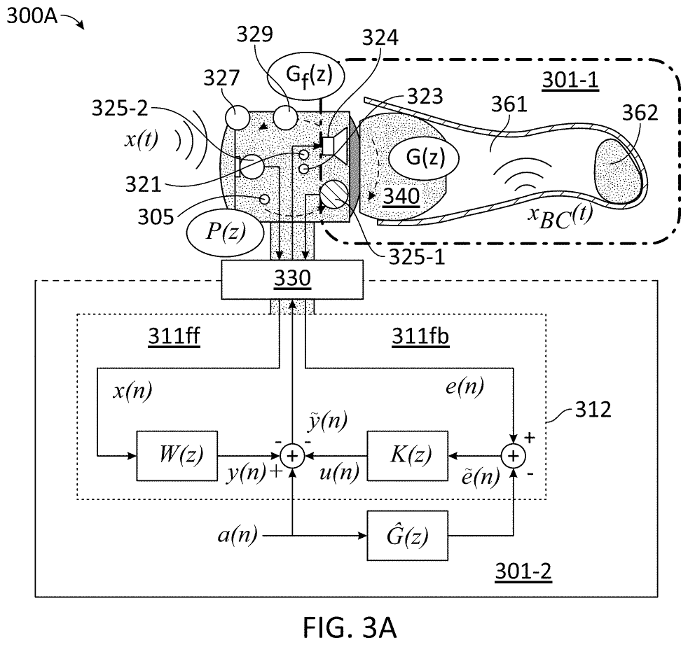

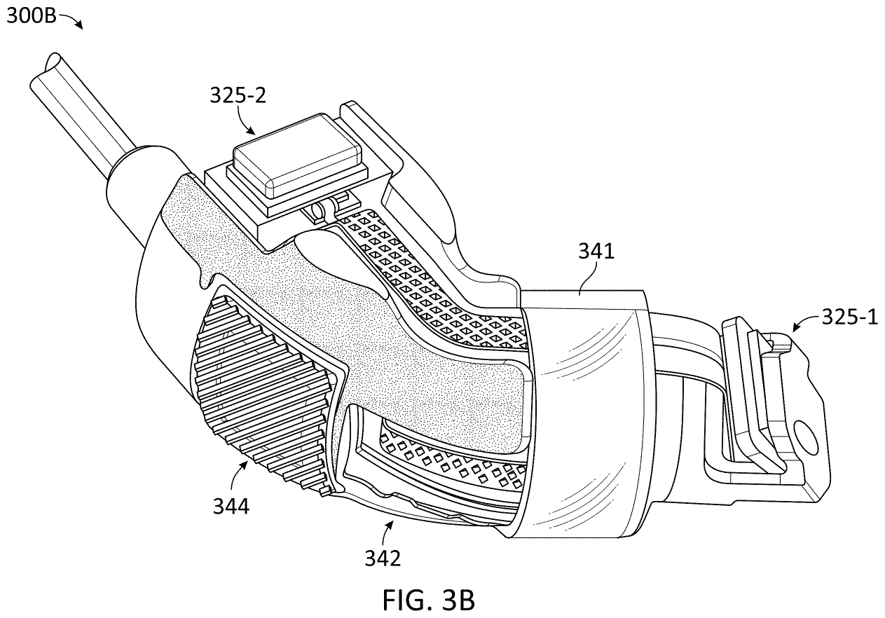

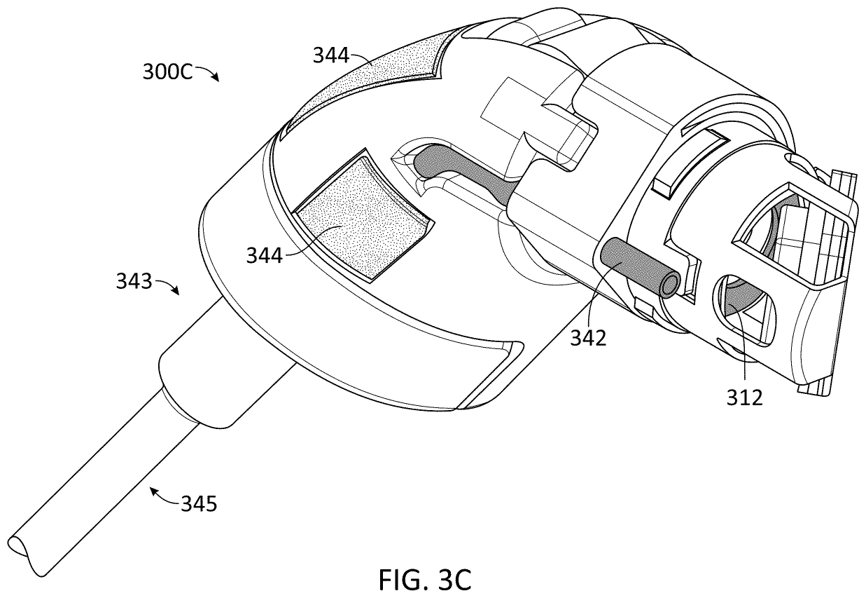

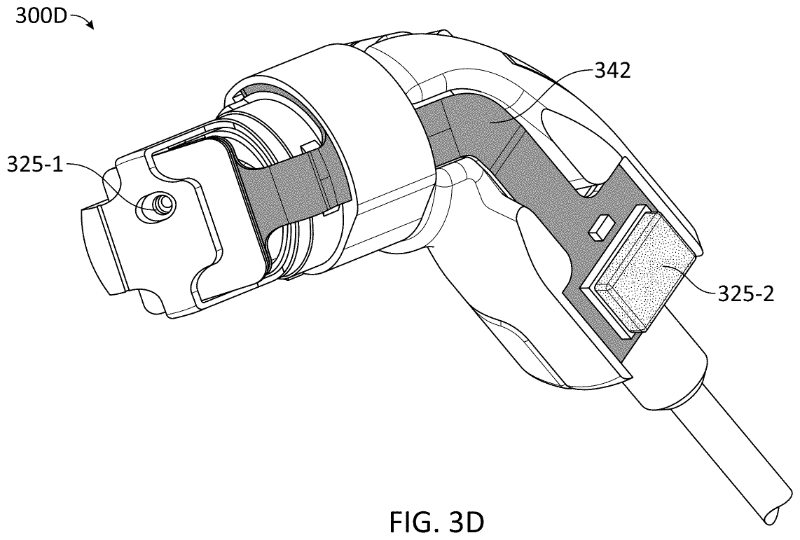

FIGS. 3A-3D illustrate different embodiments of an in-ear monitor (IEM) 300A, 300B, 300C, and 300D (hereinafter, collectively referred to as “IEMs 300”), according to some embodiments. IEMs 300 may include a front end 301-1 including sensors and open to ear canal 361 and ear drum 362, and a back end 301-2 including a processor 312. IEMs 300 may include sensors such as: an electrode 305 to sense electrical signals, acoustic sensors 325-1 and 325-2 (e.g., collectively referred hereinafter, as “microphones 325”), motion sensors 327 (e.g., accelerometers, contact microphones, inertial motion units —IMUs, and the like), temperature sensors 329, and optical sensors including an emitter 321 and a detector 323 (e.g., LEDs and PDs in PPG sensors, functional near-infrared spectroscopy fNIRS sensors —Fourier transform based, spectroscopic based-). Electrodes 305 may include bio-potential electrodes for applications such as EEG, ECG, EOG, and EDA). In some cases, the in-ear fixture 340 (also known as “eartip”) may be entirely made out of soft conductive materials. Accordingly, the entire eartip will be conductive and will act as a soft electrode. In addition, processor 312 may handle at least some of the operations for signal acquisition and control of components and sensors 321, 323, 324 (a speaker), 325-1 (internal microphone), 325-2 (external microphone, hereinafter, collectively referred to as “microphones 325”), 327, and 329 via a digital-to-analog and/or analog-to-digital converter (DAC/ADC) 330. Processor 312 may include a feedforward stage 311ff and a feedback stage 311fb that cooperate to process the signal from the sensors: noise reduction, balancing, filtering, and amplification.

In some embodiments, electrodes 305 include a contact electrode configured to transmit a current from the skin in the ear canal of the user. In some embodiments, an electrode 305 is coated with at least one of a gold layer, a silver layer, a silver chloride layer, or a combination thereof. In some embodiments, electrodes 305 include a capacitive coupling electrode disposed sufficiently close, but not in contact, with the user's skin. In some embodiments, IEMs 300 further include at least a second electrode 305 mounted on in-ear fixture 340, the second electrode 305 configured to receive a second electronic signal from the skin in ear canal 361. In some cases, the in-ear fixture 340 may be entirely made out of soft conductive materials (e.g., conductive polymers, conductive adhesives, conductive paints, etc.). Accordingly, in some embodiments the entire eartip will be conductive and will act as a soft electrode to collect electrical signals from the skin of the ear-canal. In some embodiments, processor 312 is configured to select the first electronic signal when a quality of the first electronic signal is higher than a pre-selected threshold. In some embodiments, processor 312 is configured to reduce a noise background from the first electronic signal with the second electronic signal. In some embodiments, processor 312 is configured to determine a heart rate of the user from the first electronic signal. In some embodiments, processor 312 is configured to determine a brain activity from the first electronic signal that corresponds to an acoustic stimulus received in the external microphone.

IEMs 300 in the AR headset or smart glasses may include an in-ear fixture 340 configured to hermetically seal an ear canal of a user, a first electrode 305 mounted on in-ear fixture 340 and configured to receive a first electronic signal from a skin in ear canal 361, and an internal microphone 325-1 coupled to receive an internal acoustic signal, propagating through ear canal 361. An acoustic front end includes internal microphone 325-1 configured to detect acoustic waves (x_{BC}(t)) propagated through ear canal 361 and generated by the inner body (e.g., heart rate at about <100 Hz, breathing rate at about 50-1000 Hz, and other sounds in the laryngeal cavity). An external microphone 325-2 is coupled to receive an external acoustic signal x(t), propagating through an environment of the user. In some embodiments, the internal signal x_{BC}(t) in conjunction with the external signal x(t) may be used in acoustic procedures such as audio streaming, hear-through, active noise cancelation (ANC), hearing corrections, virtual presence and spatial audio, call services, and the like. In some embodiments, at least some of the above processes are performed in conjunction between left-ear and right-ear IEM monitors 300.

IEM 300B includes a sealing gasket 341 that separates the inner portion of ear canal 361 from the environment, leaving a back-volume vent including an acoustically resistive mesh 344 for a pressure equalizer (PEQ) tube 342 to vent into resistive mesh 344 (also shown in IEM 300C). The sealed cavity may enable breathing and heart rate monitoring (e.g., isolating the signal from internal acoustic microphone 325-1) at low power usage and with a small form factor.

IEM 300C illustrates processor circuit 312 to identify a cardiovascular condition or a neurologic condition of the user, based on at least one of a first electronic signal, an internal acoustic signal, and an external acoustic signal (e.g., from microphones 325). Some embodiments may include a down cable 345 to electrically couple the IEM with the VR headset or smart glasses, including a strain relief 343.

IEM 300D illustrates a flexible, printed circuit board (FPCB) 342 that provides internal electrical connectivity to the different components and sensors 321, 323, 325, 327, and 329.

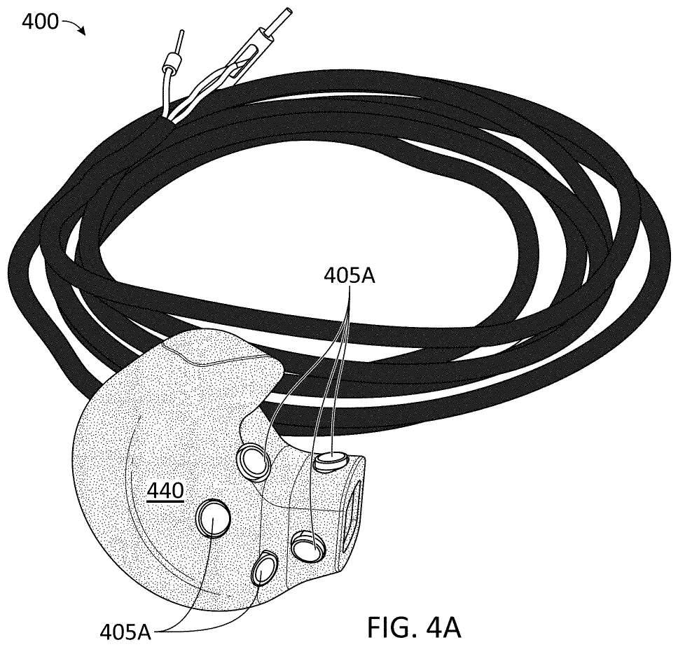

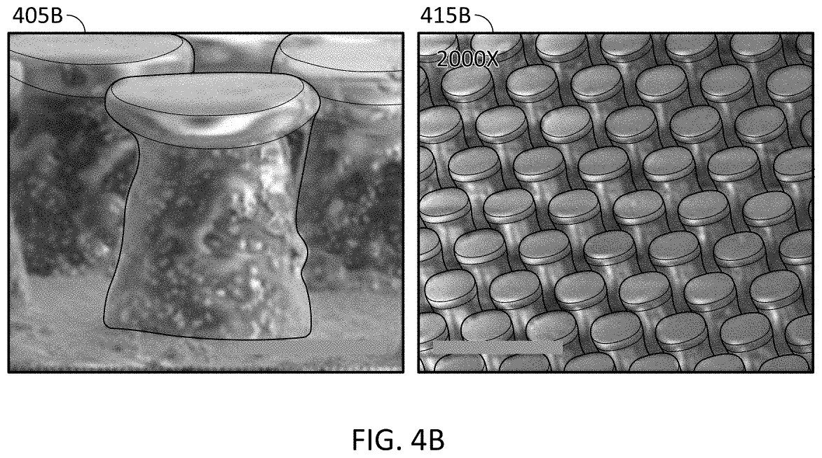

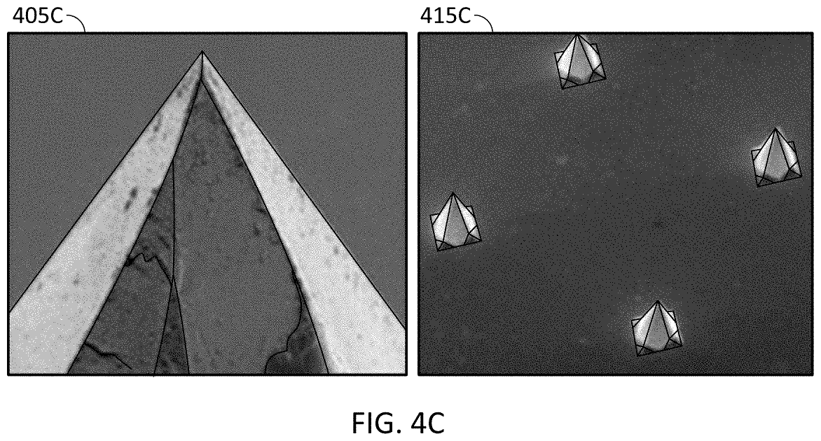

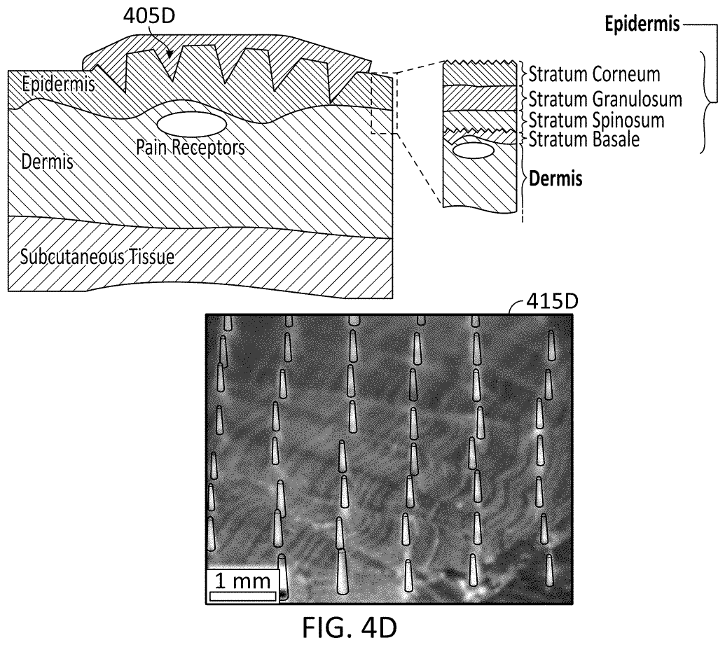

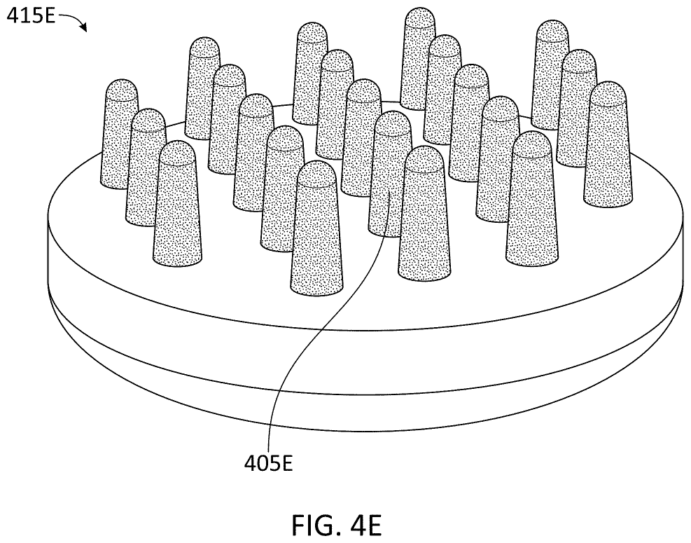

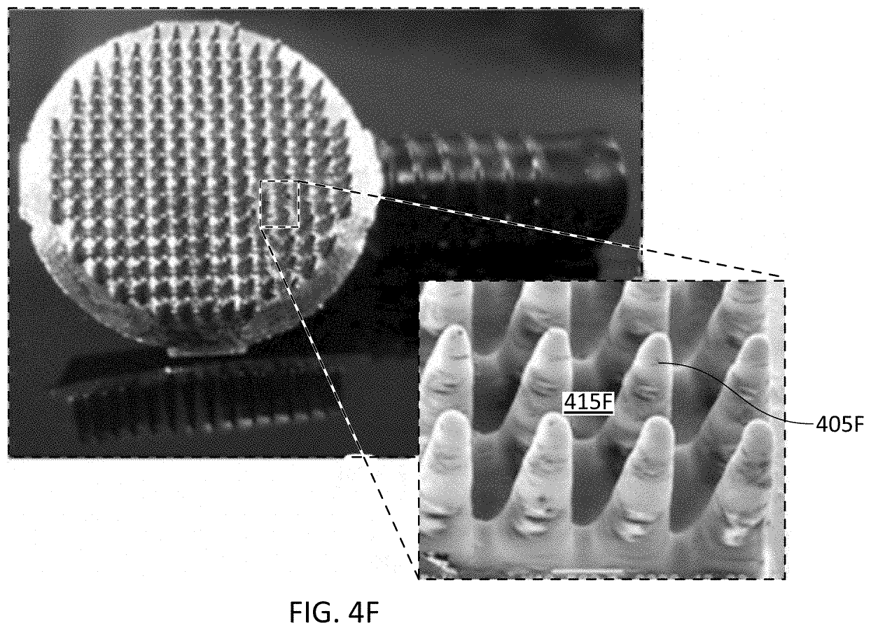

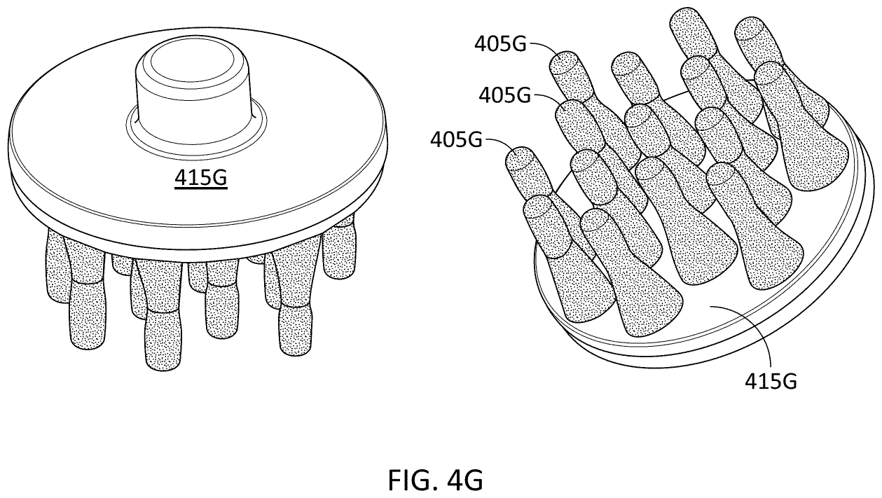

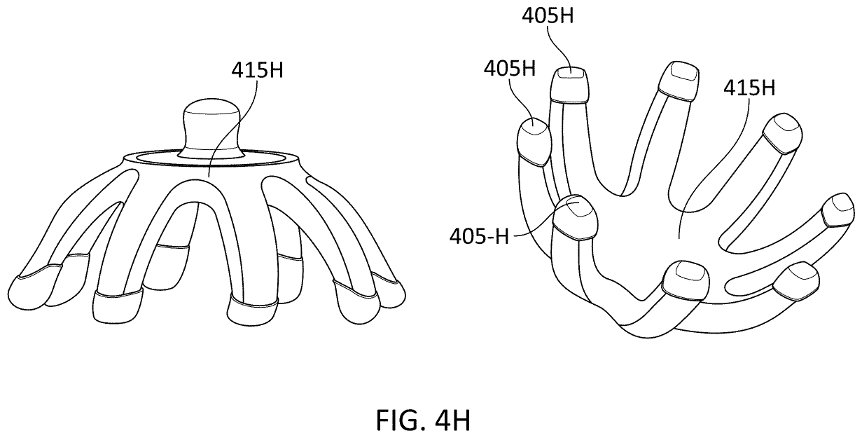

FIGS. 4A-4H illustrate multiple electrodes 405A, 405B, 405C, 405D, 405E, 405F, 405G, and 405H (hereinafter, collectively referred to as “electrodes 405”) configured to be disposed across an IEM 400 for EEG, ECG, EDA, and EOG sensing, according to some embodiments. Electrodes 405 may be formed of a conductor alloy and configured to be in contact with the user's skin in the ear canal and identify electrical signals from the body (e.g., generated by neural/muscular activity). In some embodiments, electrodes 405 may be configured to capacitively detect electrical signals from the user's body near the inner ear (not necessarily in contact with the user's skin).

In some embodiments, multiple electrodes may be included to have redundant information in case the signal from one or more of the electrodes is not strong enough or missing altogether (e.g., contact is faulty or non-existent at one or more points). In some embodiments, the multiplicity of electrodes may be used for determining spatial distribution of electrical activity inside the ear canal. This is shown with electrodes 405A disposed on in-ear fixture 440, and with electrode arrays 415B, 415C, 415D, 415E, 415F, 415G, and 415H (hereinafter, collectively referred to as “electrode arrays 415”).

Different types of electrodes 405 can be used, such as: dry electrodes; dry contact electrode and dry non-contact (capacitive) electrodes; stretchable soft electrodes; passive, active, dry, and sponge (R-NET) electrodes. In some embodiments, wearable EEG applications may include silver-coated polymer bristles, dry foam electrodes, polymer electrodes made of polydimethylsiloxane (PDMS) or polyurethane, and comb-shaped polymer electrodes which provide soft contact to the skin while still providing low electrode-to-skin contact impedance in the order of 20 kΩ to 500 kΩ. In some embodiments, earwax can be beneficial for dry electrodes to reduce electrode-to-skin contact impedance. In some cases, gels may be applied to the electrodes to enhance the conductivity and reduce the electrode-to-skin contact impedance. Typical values of the electrode-skin impedance range between 150 to 200 kΩ and 20 to 70 KΩ before and after gel applications, respectively.

FIGS. 4B-4H illustrate different embodiments of electrodes 405 for an in-ear monitor including novel electrode materials, and microstructure designs (cf. electrode arrays 415) to overcome hair and dead skin barriers, according to some embodiments. Materials and fabrication techniques as disclosed herein improve the signal quality by increasing surface area to reduce contact impedance (e.g., reducing the series resistance) and to increase parallel capacitance of the electronic coupling. Different materials may improve interface properties and increase effective surface area of electrodes, such as intrinsically conductive polymers (e.g., PEDOT:PSS, polyaniline, and polypyrrole) and extrinsically conducting polymers such as conductive material (nanowire, nanotube, nanoparticle) filled polymers and blended conductive polymers. For example, polystyrene sulfonate (PEDOT:PSS) is a polymer mixture of two ionomers (e.g., transparent electrode material). Some embodiments may include microneedle electrodes (heights range up to 500 um) that can go through the outermost SC layer and reduce the interface impedance and noise without causing pain due to the location of pain receptors (>1 mm from skin surface).

In some embodiments, electrodes 405 may be used to assess the fit of an IEM sensor into the user's ear. For example, the fit of the IEM sensor may include a measure of electrical contact. In some embodiments, individual or groups of individual electrodes 405 in electrode arrays 415 may be individually sensed or driven with an electrical current, or both, to assess fit of the IEM sensor within the user's ear, and to measure changes in the electric properties or signals within the user's skin. In some embodiments, electrode arrays 415 are formed on a compliant (e.g., flexible) circuit board such that the electrodes 405 may conform to the user's ear. Compliance may include the ability to form approximate Zernike shapes to maximize contact with the skin. In electrode arrays 415, the area surrounding the electrode posts may capture organic materials and be difficult to clean. To address this issue, in some embodiments the area surrounding electrode arrays 415 may be filled with an elastomeric material. In some embodiments, the elastomeric material may be in the form of a membrane approximately parallel with the surface of the needle, where there is a fluid such as air between the membrane and the electrodes' substrate. In some embodiments, the elastomeric material includes a compressible material, such as a closed or open cell foam. In some embodiments, the elastomeric material includes a material with a low modulus and is able to be stretched between the electrode needles. In some embodiments, the elastomeric material may include a silicore, such as polydimethyl silane polyurethane, rubbers, and the like.

FIG. 4B illustrates electrode 405B made of AgLMP, and arrays 415B for skin adhesion.

FIG. 4C illustrates electrode 405C having with a 500 μm layer of Gold coated on a Silicon substrate, forming an array 415C.

FIG. 4D illustrates an electrode 405D including a multi-needle array 415D that increases a surface contact with the skin in the ear canal of the user, reduces a resistivity, and secures electrode 405D to the skin in the ear canal of the user. Each of the needles in multi-needle array 415D may include a 350 μm layer of Silver-titanium coated on PLA.

FIG. 4E illustrates electrode 405E having carbon nanotube (CNT) doped EPDM at various heights, and forming electrode array 415E.

FIG. 4F illustrates electrode 405F including Silver coated bristles and forming electrode array 415F.

FIG. 4G illustrates electrode 405G having a 3 mm Gold coated 3D print and forming electrode array 415G.

FIG. 4H illustrates electrode 405H having Ag/AgCl Ink and forming electrode array 415H.

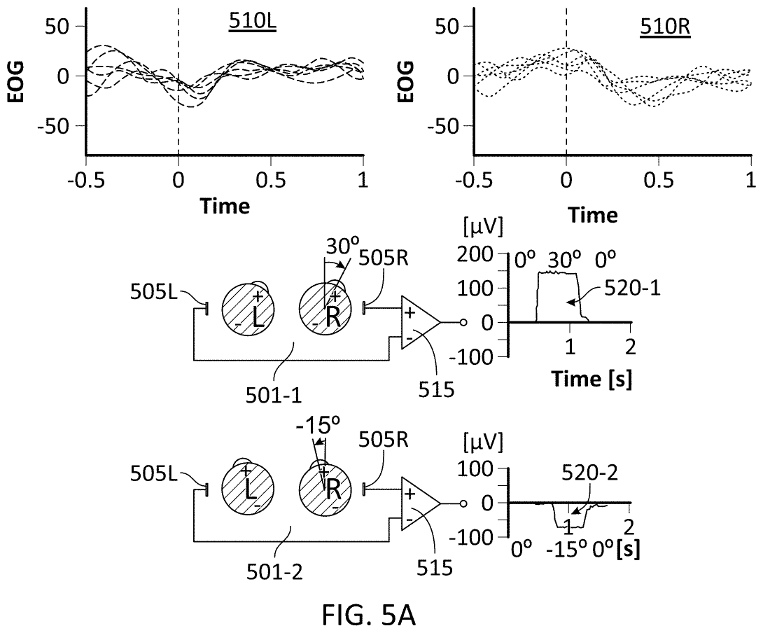

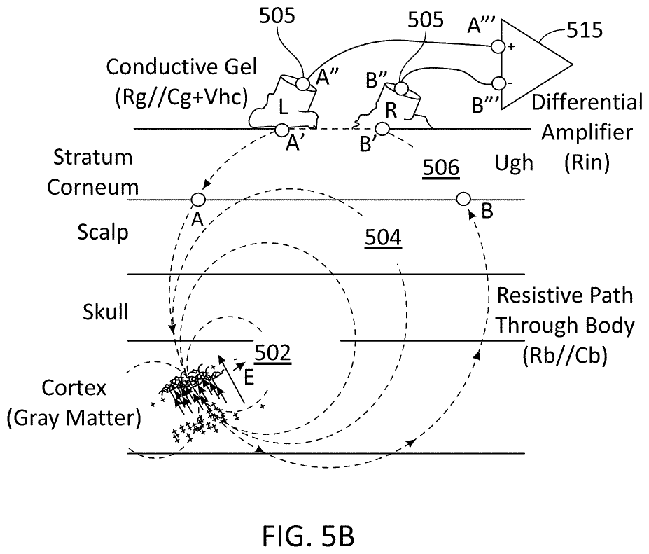

FIGS. 5A-5B illustrate EOG waveforms 510L and 510R (hereinafter, collectively referred to as “waveforms 510”) for gaze estimation with IEM 501, according to some embodiments. Waveforms 510 indicate an EOG signal amplitude (ordinate) as a function of time (abscissae). In some embodiments, a processor in the AR headset or smart glasses is configured to synchronize waveform 510L with a first electronic signal from one ear (e.g., left ear) of the user and waveform 510R with a second electronic signal from an opposite ear (e.g., right ear) of the user and to determine a gaze direction based on a comparison between the first electronic signal (e.g., waveforms 510L) and the second electronic signal (e.g., waveforms 510R). In some embodiments, the electrodes will be distributed on the glasses contact areas (such as areas where the glasses sit on top of the ears and on the nose, i.e., nose pad area) to capture the EOG information on a glasses form-factor.

In some embodiments, differential EOG signals 520-1 and 520-2 (hereinafter, collectively referred to as “differential signals 520”) are set up for gaze estimation with an IEM. Differential signals 520 are provided with a differential amplifier 515. Electrodes 505L and 505R from both IEMs (e.g., left and right, hereinafter, collectively referred to as “electrodes 505”) may capture the motion of the user's left and right eyes by capturing the change in electrical signal produced when the user's eyes move in a given direction.

The human eye acts as an electric dipole aligned along the retina-pupil axis (indicated, arbitrarily, with ‘+’ and ‘−’ signs in the figure). The dipole is conformed by potential differences across different tissue layers such as the nervous web, at the brain cortex 502, the skull 504, and the stratum corneum 506. Accordingly, when the user's eye moves in a given gaze direction, the electric field of the eye dipoles presents a different potential to electrodes 505, thus altering a measured waveform (and differential signals 520). In some embodiments, EOG signals may be further sensitive to vertical gaze estimation. In some embodiments, EOG waveforms 510 may be more sensitive to horizontal gaze movements.

For example, when the eyes move about 30° to the right, a positive trend in the waveform is observed between 0 s-1 s (which can be up to ˜150 μV, cf. differential signal 520-1). When the eyes move about 15° to the left, a negative trend is observed between 0 s-1 s (which can get down to ˜−75 μV, cf. differential signal 520-2).

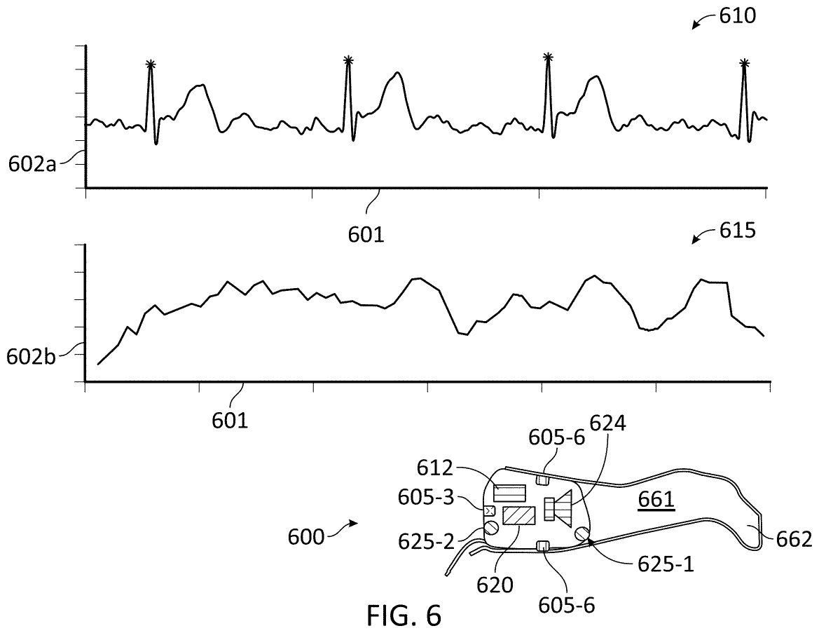

FIG. 6 illustrates chart 600 including an ECG waveform 610 and a heart rate variability (HRV) waveform 615 measured with an IEM, according to some embodiments. HRV waveform 615 is measured from ECG waveform 610, then tracking the heartrate over time to render a variability value for waveform 615. Chart 600 includes time in the abscissae 601 and signal amplitude (e.g., Voltage 602a, or beats per minute 602b) in the ordinates. Waveform 610 may be collected from any one of electrodes 605-3 (“world-facing” electrode), and 605-6. IEM 600 may include a loudspeaker 624, an internal microphone 625-1 facing ear drum 662 in ear canal 661, an external microphone 625-2, a processor 612, and a memory 620. In some embodiments, an IEM may include world-facing electrode 605-3 configured to receive a user touch and close a bodily loop that includes the heart, to obtain ECG measurements.

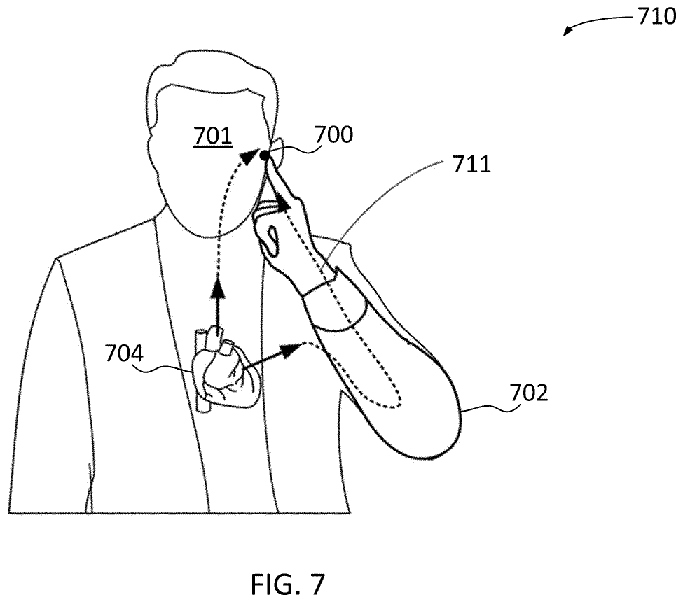

FIG. 7 illustrates a gesture 710 for an on-demand ECG capture system from an IEM device, according to some embodiments. A touch gesture 710 can register an in-ear ECG by closing an electrical loop 711 between the arm 702, IEM 700, and the heart 704. Accordingly, electrical pulses generated at the heart muscle 704 can reach IEM 700 simply by having user 701 touch a “world-facing electrode” in IEM 700 with a finger. Accordingly, no less than two electrodes on each in-ear device 700 (left and right) are included, and at least one of the electrodes remains in contact with the skin (e.g., inside the ear canal, touching the ear canal walls). A second in-ear electrode may be a “world-facing electrode.” Accordingly, the second electrode may not be in contact with the skin, and when the user needs to have a highly accurate ECG signal with very low noise, the user brings their finger and touches the second, “world-facing electrode,” so to close the loop through the heart and create a high-SNR, ECG waveform.

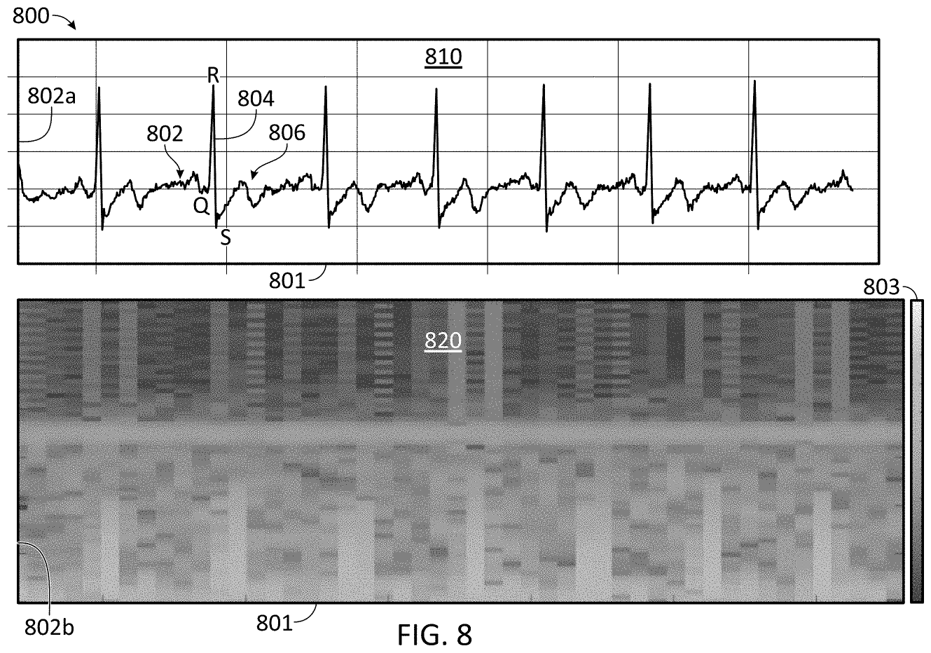

FIG. 8 illustrates charts 800 including in-ear ECG waveform 810 and its spectral decomposition 820, according to some embodiments. The abscissae in charts 800 is time 801, and the ordinates indicate signal value 802a and frequency 802b. A color scale 803 indicates amplitude in spectral decomposition 820. When the user touches the world-facing electrode (cf. gesture 710), ECG waveform 810 is captured with a bio-potential chip (e.g., 8-channel 24-bit processor with a sampling rate of 250 Hz). In some other embodiments, higher sampling rates such as 1000 Hz or 2000 Hz may be used; ECG features such as p-wave 802, QRS-complex 804, and T-wave 806 are obtained using these electrode configurations.

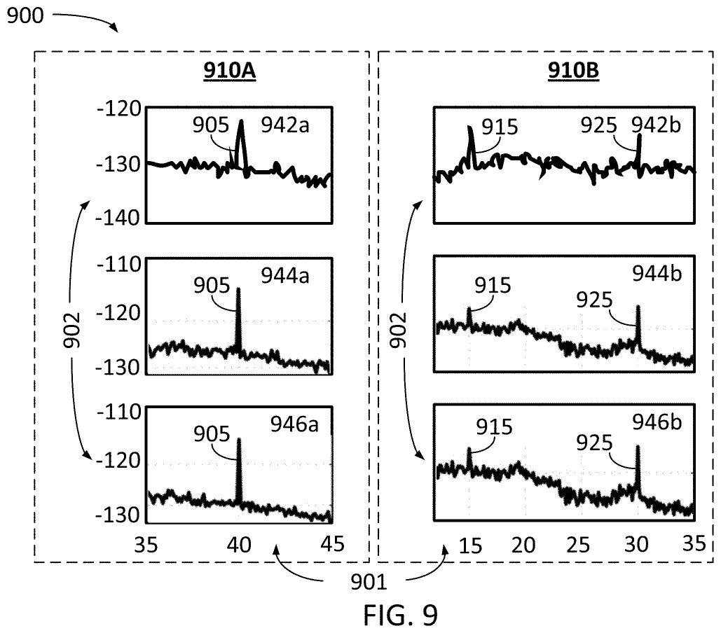

FIG. 9 illustrates a hearing threshold estimation 900 using an IEM, according to some embodiments. ASSR spectra 910A and steady state visually evoked potential (SSVEP) 910B are illustrated, with amplitude 902 in the ordinates (e.g., dBm) and modulation frequency 901 in the abscissae (e.g., Hz). In-ear spectra 942a and 942b (hereinafter, collectively referred to as “ear spectra 942”) are compared to mastoid spectra 944a and 944b (hereinafter, collectively referred to as “mastoid spectra 944”), and on-scalp 946a and 946b (hereinafter, collectively referred to as “scalp spectra 946”). ASSR spectra 910A shows a frequency peak at 40 Hz which corresponds to the frequency of the amplitude modulation (AM) of a 1000 Hz tone. The magnitude of the peak is similar to on-scalp and mastoid (M1) EEG electrodes. SSVEP spectra 910B may be induced by illuminating the user with an LED blinking at 15 Hz. A clear peak 915 is observed at the stimulus frequency of 15 Hz and also at its first harmonic 925 at 30 Hz. SSVEP can be used to obtain the health of the vision system along with the corresponding signal chain to the brain. The signal for ear spectra 942 is weaker than mastoid spectra 944 and scalp spectra 946 due to a larger distance between the EEG source in the occipital region (involved in SSVEP) and the ear canal, and because of smaller electrode distances within ear-EEG, according to biophysics propagation models for the human brain.

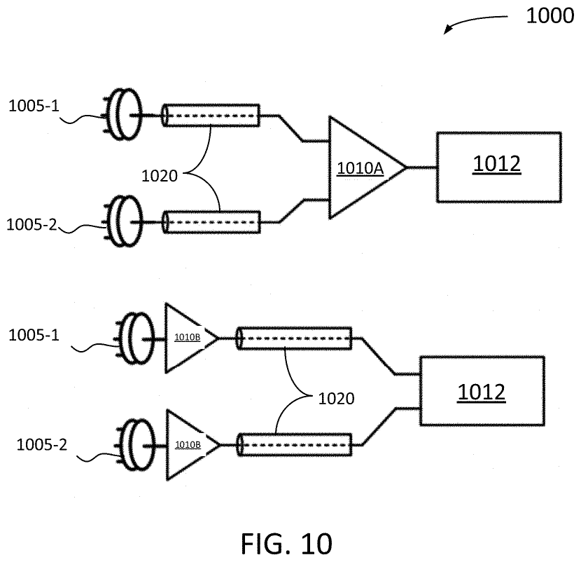

FIG. 10 illustrates a block diagram 1000 of a conventional EEG readout with an instrumental amplifier (IA) 1010A or a buffered amplifier 1010B coupled to electrodes 1005-1 and 1005-2 (hereinafter, collectively referred to “electrodes 1005”), according to some embodiments. In some embodiments, the IEM includes a second electrode 1005-2 in a second in-ear fixture, configured to receive a second electronic signal from the skin in a second ear canal of the user with parallel impedance coupling 1020. IA 1010A is configured to amplify a difference signal between electrodes 1005 and to provide the difference signal to a back-end processor 1012.

A buffered amplifier 1010B is coupled to electrodes 1005 to provide amplified electronic signals to back-end processor 1012. Amplifiers 1010A and 1010B will be collectively referred to as “amplifiers 1010.” In some embodiments, buffered amplifier 1010B locally amplifies and buffers μV-level EEG signals before driving any cabling. In some embodiments, electrodes 1005 with buffered amplifier 1010B are built-in readout circuitry desirable for wearable healthcare and lifestyle applications because of their robustness to environmental interference. Buffered amplifier 1010B can translate the voltage of the source to the voltage desirable by the load. Buffered amplifier 1010B allows a subcircuit with only low or modest current-source/sink capability to drive a load which requires more current to operate.

In some embodiments, buffered amplifier 1010B has desirably a high input impedance because of a reduced signal path length between electrodes 1005 and a first amplifier stage, and an output impedance lower than 1 Ohm, ensuring that the signal in the cable is fully insensitive to interference. A low output impedance mitigates cable motion artifacts, which eliminates the need of shielded cables, and enables the use of high impedance dry electrodes 1005 for greater user comfort. In some embodiments, buffered amplifier 1010B amplifies the EEG signal and decreases the influence of ambient electrical noise. Buffered amplifier 1010B also reduces the routing to electrodes 1005. This reduces the cost of IEMs and noise pickup.

Amplifiers 1010 may desirably have input reference noise of less than 1 μV and an input impedance higher than 100 Mega Ohm (10⁶ Ohm).

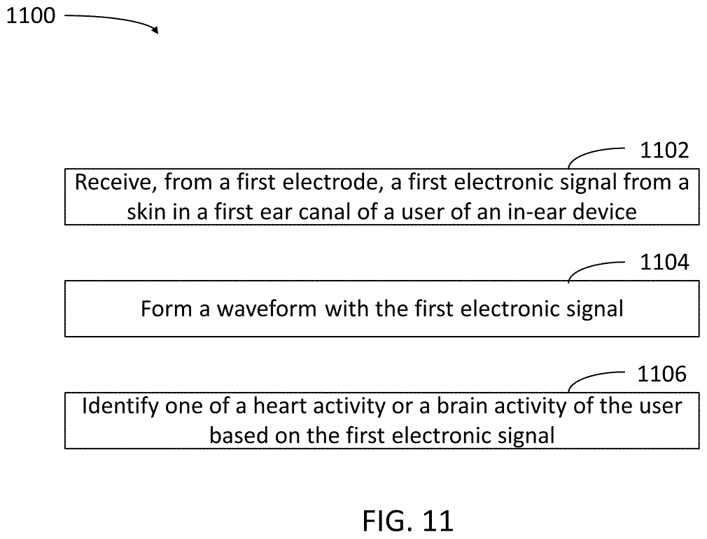

FIG. 11 is a flow chart illustrating steps in a method 1100 for using electrodes in an in-ear monitor for assessing the health of a user of a headset or smart glasses, according to some embodiments. In some embodiments, at least one or more of the steps in method 1100 may be performed by a processor executing instructions stored in a memory in either one of smart glasses or other wearable devices on a user's body part (e.g., head, arm, wrist, leg, ankle, finger, toe, knee, shoulder, chest, back, and the like). In some embodiments, at least one or more of the steps in method 1100 may be performed by a processor executing instructions stored in a memory, wherein either the processor or the memory (cf., processors 112, 312, 612, and 1012 and memories 120 and 620), or both, are part of a mobile device for the user, a remote server or a database, communicatively coupled with each other via a network (e.g., client devices 110, server 130, and network 150). Moreover, the mobile device, the smart glasses, and the wearable devices may be communicatively coupled with each other via a wireless communication system and protocol (e.g., communications module 118, radio, Wi-Fi, Bluetooth, near-field communication —NFC- and the like). In some embodiments, a method consistent with the present disclosure may include one or more steps from method 1100 performed in any order, simultaneously, quasi-simultaneously, or overlapping in time.

Step 1102 includes receiving, from a first electrode, a first electronic signal from a skin in a first ear canal of a user of an in-ear device. In some embodiments, step 1102 includes receiving, from a second electrode, a second electronic signal from the skin in the first ear canal of the user of the in-ear device and removing an interference from the first electronic signal with the first electronic signal. In some embodiments, step 1102 further includes receiving, from a second electrode, a second electronic signal from the skin in a second ear canal of the user of the in-ear device, identifying an eye gaze direction based on the first electronic signal and the second electronic signal. In some embodiments, step 1102 includes receiving an acoustic signal from an external microphone in the in-ear device in response to an acoustic stimulus correlating the acoustic signal with the first electronic signal. In some embodiments, step 1102 includes receiving, from an optical sensor, an optical signal.

Step 1104 includes forming a waveform with the first electronic signal.

Step 1106 includes identifying one of a heart activity or a brain activity of the user based on the first electronic signal. In some embodiments, step 1106 includes identifying an eye gaze direction based on the first electronic signal and the second electronic signal. In some embodiments, step 1106 further includes assessing a user response to the acoustic stimulus based on the brain activity and the acoustic stimulus. In some embodiments, step 1106 includes performing a spectral analysis on the waveform to identify a p-wave, a QRS-complex, and a T-wave complex in an electro-cardiogram. In some embodiments, identifying the heart activity in step 1106 includes correlating the first electronic signal with the optical signal. In some embodiments, step 1106 further includes measuring a change in an electric property within a user's skin and assessing a fit of the in-ear device within a user's ear based on the change in the electrical property.

### Hardware Overview

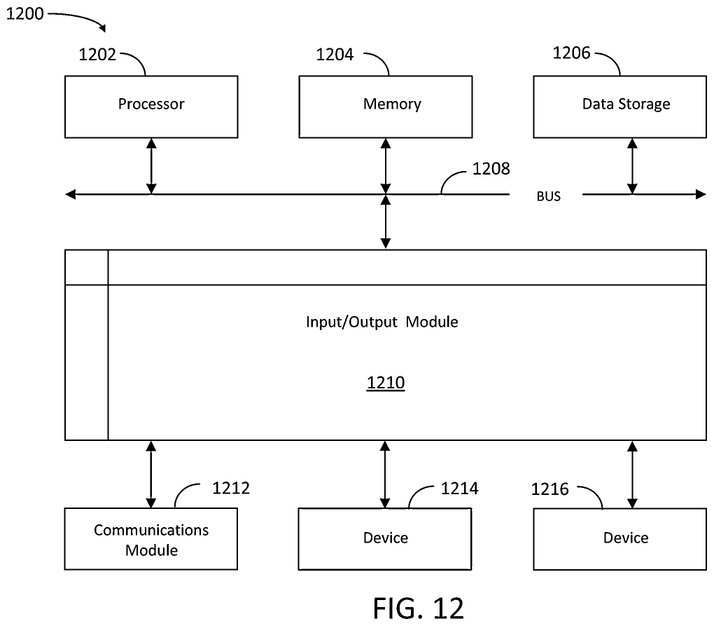

FIG. 12 is a block diagram illustrating an exemplary computer system 1200 with which headsets and other client devices 110, and method 1100 can be implemented, according to some embodiments. In certain aspects, computer system 1200 may be implemented using hardware or a combination of software and hardware, either in a dedicated server, or integrated into another entity, or distributed across multiple entities. Computer system 1200 may include a desktop computer, a laptop computer, a tablet, a phablet, a smartphone, a feature phone, a server computer, or otherwise. A server computer may be located remotely in a data center or be stored locally.

Computer system 1200 includes a bus 1208 or other communication mechanism for communicating information, and a processor 1202 (e.g., processor 112) coupled with bus 1208 for processing information. By way of example, the computer system 1200 may be implemented with one or more processors 1202. Processor 1202 may be a general-purpose microprocessor, a microcontroller, a Digital Signal Processor (DSP), an Application Specific Integrated Circuit (ASIC), a Field Programmable Gate Array (FPGA), a Programmable Logic Device (PLD), a controller, a state machine, gated logic, discrete hardware components, or any other suitable entity that can perform calculations or other manipulations of information.

Computer system 1200 can include, in addition to hardware, code that creates an execution environment for the computer program in question, e.g., code that constitutes processor firmware, a protocol stack, a database management system, an operating system, or a combination of one or more of them stored in an included memory 1204 (e.g., memory 120), such as a Random Access Memory (RAM), a flash memory, a Read-Only Memory (ROM), a Programmable Read-Only Memory (PROM), an Erasable PROM (EPROM), registers, a hard disk, a removable disk, a CD-ROM, a DVD, or any other suitable storage device, coupled with bus 1208 for storing information and instructions to be executed by processor 1202. The processor 1202 and the memory 1204 can be supplemented by, or incorporated in, special purpose logic circuitry.

The instructions may be stored in the memory 1204 and implemented in one or more computer program products, e.g., one or more modules of computer program instructions encoded on a computer-readable medium for execution by, or to control the operation of, the computer system 1200, and according to any method well known to those of skill in the art, including, but not limited to, computer languages such as data-oriented languages (e.g., SQL, dBase), system languages (e.g., C, Objective-C, C++, Assembly), architectural languages (e.g., Java, .NET), and application languages (e.g., PHP, Ruby, Perl, Python). Instructions may also be implemented in computer languages such as array languages, aspect-oriented languages, assembly languages, authoring languages, command line interface languages, compiled languages, concurrent languages, curly-bracket languages, dataflow languages, data-structured languages, declarative languages, esoteric languages, extension languages, fourth-generation languages, functional languages, interactive mode languages, interpreted languages, iterative languages, list-based languages, little languages, logic-based languages, machine languages, macro languages, metaprogramming languages, multiparadigm languages, numerical analysis, non-English-based languages, object-oriented class-based languages, object-oriented prototype-based languages, off-side rule languages, procedural languages, reflective languages, rule-based languages, scripting languages, stack-based languages, synchronous languages, syntax handling languages, visual languages, with languages, and xml-based languages. Memory 1204 may also be used for storing temporary variable or other intermediate information during execution of instructions to be executed by processor 1202.

A computer program as discussed herein does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, subprograms, or portions of code). A computer program can be deployed to be executed on one computer or on multiple computers that are located at one site or distributed across multiple sites and interconnected by a communication network. The processes and logic flows described in this specification can be performed by one or more programmable processors executing one or more computer programs to perform functions by operating on input data and generating output.

Computer system 1200 further includes a data storage device 1206 such as a magnetic disk or optical disk, coupled with bus 1208 for storing information and instructions. Computer system 1200 may be coupled via input/output module 1210 to various devices. Input/output module 1210 can be any input/output module. Exemplary input/output modules 1210 include data ports such as USB ports. The input/output module 1210 is configured to connect to a communications module 1212. Exemplary communications modules 1212 include networking interface cards, such as Ethernet cards and modems. In certain aspects, input/output module 1210 is configured to connect to a plurality of devices, such as an input device 1214 and/or an output device 1216. Exemplary input devices 1214 include a keyboard and a pointing device, e.g., a mouse or a trackball, by which a consumer can provide input to the computer system 1200. Other kinds of input devices 1214 can be used to provide for interaction with a consumer as well, such as a tactile input device, visual input device, audio input device, or brain-computer interface device. For example, feedback provided to the consumer can be any form of sensory feedback, e.g., visual feedback, auditory feedback, or tactile feedback; and input from the consumer can be received in any form, including acoustic, speech, tactile, or brain wave input. Exemplary output devices 1216 include display devices, such as an LCD (liquid crystal display) monitor, for displaying information to the consumer.

According to one aspect of the present disclosure, headsets and client devices 110 can be implemented, at least partially, using a computer system 1200 in response to processor 1202 executing one or more sequences of one or more instructions contained in memory 1204. Such instructions may be read into memory 1204 from another machine-readable medium, such as data storage device 1206. Execution of the sequences of instructions contained in main memory 1204 causes processor 1202 to perform the process steps described herein. One or more processors in a multi-processing arrangement may also be employed to execute the sequences of instructions contained in memory 1204. In alternative aspects, hard-wired circuitry may be used in place of or in combination with software instructions to implement various aspects of the present disclosure. Thus, aspects of the present disclosure are not limited to any specific combination of hardware circuitry and software.

Various aspects of the subject matter described in this specification can be implemented in a computing system that includes a back end component, e.g., a data server, or that includes a middleware component, e.g., an application server, or that includes a front end component, e.g., a client computer having a graphical consumer interface or a Web browser through which a consumer can interact with an implementation of the subject matter described in this specification, or any combination of one or more such back end, middleware, or front end components. The components of the system can be interconnected by any form or medium of digital data communication, e.g., a communication network. The communication network can include, for example, any one or more of a LAN, a WAN, the Internet, and the like. Further, the communication network can include, but is not limited to, for example, any one or more of the following network topologies, including a bus network, a star network, a ring network, a mesh network, a star-bus network, tree or hierarchical network, or the like. The communications modules can be, for example, modems or Ethernet cards.

Computer system 1200 can include clients and servers. A client and server are generally remote from each other and typically interact through a communication network. The relationship of client and server arises by virtue of computer programs running on the respective computers and having a client-server relationship to each other. Computer system 1200 can be, for example, and without limitation, a desktop computer, laptop computer, or tablet computer. Computer system 1200 can also be embedded in another device, for example, and without limitation, a mobile telephone, a PDA, a mobile audio player, a Global Positioning System (GPS) receiver, a video game console, and/or a television set top box.

The term “machine-readable storage medium” or “computer-readable medium” as used herein refers to any medium or media that participates in providing instructions to processor 1202 for execution. Such a medium may take many forms, including, but not limited to, non-volatile media, volatile media, and transmission media. Non-volatile media include, for example, optical or magnetic disks, such as data storage device 1206. Volatile media include dynamic memory, such as memory 1204. Transmission media include coaxial cables, copper wire, and fiber optics, including the wires forming bus 1208. Common forms of machine-readable media include, for example, floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM, a FLASH EPROM, any other memory chip or cartridge, or any other medium from which a computer can read. The machine-readable storage medium can be a machine-readable storage device, a machine-readable storage substrate, a memory device, a composition of matter affecting a machine-readable propagated signal, or a combination of one or more of them.

As used herein, the phrase “at least one of” preceding a series of items, with the terms “and” or “or” to separate any of the items, modifies the list as a whole, rather than each member of the list (e.g., each item). The phrase “at least one of” does not require selection of at least one item; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

The word “exemplary” is used herein to mean “serving as an example, instance, or illustration.” Any embodiment described herein as “exemplary” is not necessarily to be construed as preferred or advantageous over other embodiments. Phrases such as an aspect, the aspect, another aspect, some aspects, one or more aspects, an implementation, the implementation, another implementation, some implementations, one or more implementations, an embodiment, the embodiment, another embodiment, some embodiments, one or more embodiments, a configuration, the configuration, another configuration, some configurations, one or more configurations, the subject technology, the disclosure, the present disclosure, other variations thereof and alike are for convenience and do not imply that a disclosure relating to such phrase(s) is essential to the subject technology or that such disclosure applies to all configurations of the subject technology. A disclosure relating to such phrase(s) may apply to all configurations, or one or more configurations. A disclosure relating to such phrase(s) may provide one or more examples. A phrase such as an aspect or some aspects may refer to one or more aspects and vice versa, and this applies similarly to other foregoing phrases.

A reference to an element in the singular is not intended to mean “one and only one” unless specifically stated, but rather “one or more.” Pronouns in the masculine (e.g., his) include the feminine and neuter gender (e.g., her and its) and vice versa. The term “some” refers to one or more. Underlined and/or italicized headings and subheadings are used for convenience only, do not limit the subject technology, and are not referred to in connection with the interpretation of the description of the subject technology. Relational terms such as first and second and the like may be used to distinguish one entity or action from another without necessarily requiring or implying any actual such relationship or order between such entities or actions. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public, regardless of whether such disclosure is explicitly recited in the above description. No claim element is to be construed under the provisions of 35 U.S.C. § 112, sixth paragraph, unless the element is expressly recited using the phrase “means for” or, in the case of a method claim, the element is recited using the phrase “step for.”

While this specification contains many specifics, these should not be construed as limitations on the scope of what may be described, but rather as descriptions of particular implementations of the subject matter. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially described as such, one or more features from a described combination can in some cases be excised from the combination, and the described combination may be directed to a subcombination or variation of a subcombination.

The subject matter of this specification has been described in terms of particular aspects, but other aspects can be implemented and are within the scope of the following claims. For example, while operations are depicted in the drawings in a particular order, this should not be understood as requiring that such operations be performed in the particular order shown or in sequential order, or that all illustrated operations be performed, to achieve desirable results. The actions recited in the claims can be performed in a different order and still achieve desirable results. As one example, the processes depicted in the accompanying figures do not necessarily require the particular order shown, or sequential order, to achieve desirable results. In certain circumstances, multitasking and parallel processing may be advantageous. Moreover, the separation of various system components in the aspects described above should not be understood as requiring such separation in all aspects, and it should be understood that the described program components and systems can generally be integrated together in a single software product or packaged into multiple software products.

The title, background, brief description of the drawings, abstract, and drawings are hereby incorporated into the disclosure and are provided as illustrative examples of the disclosure, not as restrictive descriptions. It is submitted with the understanding that they will not be used to limit the scope or meaning of the claims. In addition, in the detailed description, it can be seen that the description provides illustrative examples and the various features are grouped together in various implementations for the purpose of streamlining the disclosure. The method of disclosure is not to be interpreted as reflecting an intention that the described subject matter requires more features than are expressly recited in each claim. Rather, as the claims reflect, inventive subject matter lies in less than all features of a single disclosed configuration or operation. The claims are hereby incorporated into the detailed description, with each claim standing on its own as a separately described subject matter.

The claims are not intended to be limited to the aspects described herein but are to be accorded the full scope consistent with the language claims and to encompass all legal equivalents. Notwithstanding, none of the claims are intended to embrace subject matter that fails to satisfy the requirements of the applicable patent law, nor should they be interpreted in such a way.

What is claimed is:

1. A device, comprising:
an in-ear fixture configured to fit in an ear canal of a user;
a first electrode mounted on the in-ear fixture and configured to receive a first electronic signal from a skin in the ear canal of the user;
an internal microphone coupled to receive an internal acoustic signal, propagating through the ear canal of the user;
an external microphone coupled to receive an external acoustic signal, propagating trough an environment of the user; and
a processor that is coupled to an augmented reality headset, the processor configured to identify a cardiovascular condition, or a neurologic condition of the user based on at least one of the first electronic signal, the internal acoustic signal, and the external acoustic signal.

2. The device of claim 1, further including a second electrode mounted on an outer side of the in-ear fixture, the second electrode configured to receive a signal when the user closes a bodily loop by contacting the second electrode with a finger.

3. The device of claim 1, wherein the first electrode is a contact electrode configured to transmit a current from the skin in the ear canal of the user.

4. The device of claim 1, further including at least a second electrode mounted on the in-ear fixture, the second electrode configured to receive a second electronic signal from the skin in the ear canal of the user.

5. The device of claim 1, wherein the processor is configured to select the first electronic signal when a quality of the first electronic signal is higher than a pre-selected threshold.

6. The device of claim 1, further including a second electrode configured to receive a second electronic signal from the skin in the ear canal of the user, and the processor is configured to reduce a noise background from the first electronic signal with the second electronic signal.

7. The device of claim 1, wherein the first electrode includes multiple needles that increase a surface contact with the skin in the ear canal of the user, reduces a resistivity, and secures the first electrode to the skin in the ear canal of the user, further wherein the needles are supported by a structure that includes an elastomeric material between the needles, and the elastomeric material includes at least one of a compressible material having a very low modulus and is able to be stretched between the needles.

8. The device of claim 1, wherein the first electrode is coated with at least one of a gold layer, a silver layer, a silver chloride layer, or a combination thereof.

9. The device of claim 1, wherein the processor is configured to synchronize a waveform with the first electronic signal and a waveform with a second electronic signal from an opposite ear of the user and to determine a gaze direction based on a comparison between the first electronic signal and the second electronic signal.

10. The device of claim 1, wherein the processor is configured to determine a heart rate of the user from the first electronic signal.

11. The device of claim 1, wherein the processor is configured to determine a brain activity from the first electronic signal that corresponds to an acoustic stimulus received in the external microphone.

12. The device of claim 1, further including a second electrode in a second in-ear fixture and configured to receive a second electronic signal from the skin in a second ear canal of the user; and an instrumental amplifier with a parallel impedance coupling to the first electrode and the second electrode, and configured to amplify a difference signal between the first electronic signal and the second electronic signal and to provide the difference signal to the processor.

13. The device of claim 1, further including a buffered amplifier coupled to the first electrode and configured to provide an amplified first electronic signal to the processor.

14. The device of claim 1, further including an optical sensor configured to provide an optical signal to the processor, wherein the processor is configured to identify a cardiovascular condition of the user based on the first electronic signal and the optical signal.

15. A computer-implemented method, comprising:
receiving, from a first electrode, a first electronic signal from a skin in a first ear canal of a user of an in-ear device,
forming a waveform with the first electronic signal; and
identifying one of a heart activity or a brain activity of the user based on the first electronic signal.

16. The computer-implemented method of claim 15, further including receiving, from a second electrode, a second electronic signal from the skin in the first ear canal of the user of the in-ear device, and removing an interference from the first electronic signal with the first electronic signal.

17. The computer-implemented method of claim 15, further including receiving, from a second electrode, a second electronic signal from the skin in a second ear canal of the user of the in-ear device, and identifying an eye gaze direction based on the first electronic signal and the second electronic signal.

18. The computer-implemented method of claim 15, further including receiving an acoustic signal from an external microphone in the in-ear device in response to an acoustic stimulus; correlating the acoustic signal with the first electronic signal; and assessing a user response to the acoustic stimulus based on the brain activity and the acoustic stimulus.

19. The computer-implemented method of claim 15, wherein identifying a heart activity of the user further includes performing a spectral analysis on the waveform to identify a p-wave, a QRS-complex, and a T-wave complex in an electro-cardiogram.

20. The computer-implemented method of claim 15, further including measuring a change in an electric property within a user's skin, and assessing a fit of the in-ear device within a user's ear based on the change in the electrical property.
